(19) United States
(12) Patent Application Publication
(10) Pub. No.: US 2021/0315844 A1
(43) Pub. Date: Oct. 14, 2021

(54) ENDOXIFEN FOR THE TREATMENT OF BIPOLAR I DISORDER

(71) Applicant: Jina Pharmaceuticals, Inc., Libertyville, IL (US)

(72) Inventors: Ateeq AHMAD, Wadsworth, IL (US); Imran AHMAD, Libertyville, IL (US); Moghisuddin AHMAD, Wadsworth, IL (US); Shoukath M. ALI, Vernon Hills, IL (US); Saifuddin SHEIKH, Libertyville, IL (US)

(21) Appl. No.: 17/226,651

(22) Filed: Apr. 9, 2021

Related U.S. Application Data
(60) Provisional application No. 63/008,146, filed on Apr. 10, 2020.

(51) Int. Cl. A61K 31/138; A61K 9/28; A61K 9/00; A61P 25/18

(52) U.S. Cl. CPC A61K 31/138; A61P 25/18; A61K 9/0053; A61K 9/28

(57) ABSTRACT

A method for managing or decreasing a risk of suicidal behaviour in a patient undergoing treatment of bipolar I disorder is provided. The said method includes maintaining the therapeutically effective concentration of endoxifen by administrating a dose of 2 mg to 16 mg of endoxifen citrate in an enteric coated tablet once per day for at least 21 days.

### FIELD OF THE INVENTION

The present disclosure relates to a method for managing or decreasing a risk of suicidal behaviour in a patient undergoing treatment of bipolar I disorder, wherein the said method comprises maintaining the therapeutically effective concentration of endoxifen by administrating a dose of 2 mg to 16 mg of endoxifen citrate in an enteric coated tablet once per day for at least 21 days.

### BACKGROUND OF THE INVENTION

Bipolar disorder is currently a major health problem. Bipolar disorder is a chronic, debilitating illness that affects up to 3% of the US populations. It causes significant morbidity and imposes a burden on the society. The causes of bipolar disorder are still unknown, and no agent has been specifically developed on the basis of an understanding of the pathophysiology of the illness or mechanism of action for effective treatments.

Several drugs have been approved for treatment of bipolar disorder, such as lithium, valproate or divalproex sodium, carbamazepine, and atypical antipsychotics for the treatment of acute bipolar mania. While these drugs have provided relief for many individuals with bipolar disorder, significant issues with tolerability, efficacy, and attempt suicide or have suicidal behaviour still remain. Further, divalproex has a good tolerability but a high discontinuation rate. There is also a continuous need to observe the therapeutic dose monitoring in patients during the treatment. Short term studies on clinical samples in America showed that early-onset of bipolar I disorder is associated with slow response rate to treatment, persistent mood swings, high recurrence rate, high risk of attempted suicide and severe psychosocial disorders.

The clinicians, for example, may find themselves in situations in which better tolerated agents are less effective, and vice versa. Also, the adherence to the treatment is affected by adverse effects such as sedation, weight gain, alteration in thrombocytopenia, and thyroid disorders.

Study literature reported by Dome et al. (2019) bipolar disorders are prevalent mental health illnesses that affect about 1-5% of the total population, have a chronic course and are associated with a markedly elevated premature mortality. One of the contributors for the decreased life expectancy in bipolar disorders is suicide. The rate of suicide among bipolar disorders patients is approximately 10-30 times higher than the corresponding rate in the general population. Extant research found that up to 20% of bipolar disorders subjects end their life by suicide, and 20-60% of them attempt suicide at least one in their lifetime. With a lifetime prevalence of 1.3-5.0%, type I and type II bipolar disorders are among the most common psychiatric ailments. Patients with bipolar disorders have poor life expectancies as these patients have a decreased lifespan of about 9-17 years compared with the general population. Furthermore, some studies from different countries suggest that this mortality gap has become larger over the last decades. Although the largest number of excess death cases in bipolar disorders may be attributed to natural (e.g., due to cardiovascular diseases or diabetes) and not unnatural causes, suicide is also quite prevalent in the population of subjects with bipolar disorders. At a global scale, around 800000 suicide deaths occur every year (which corresponds to a global suicide rate of 11.4/100000/year); thus, suicide may be considered a major public health issue. Although the great majority (≈90%) of suicide cases occur among subjects with major mental—typically mood disorders, the majority of patients with mood disorders never become involved in suicidal behaviour. Accordingly, in addition to major mood disorders, other risk factors (including special clinical features of the mental illness as well as some demographic, personality and familial factors) should contribute to suicidality, which therefore should be deemed as a multi causal phenomenon.

A study reported by Song et al. (2017) on around 51000 individuals with bipolar disorder, followed from 2005 to 2013 for treatment with lithium and valproate. Stratified cox regression was used to estimate the hazard ratios of suicide-related events during treated periods compared with untreated periods. For significant associations between medication and suicide-related events, the population attributable fraction was estimated to assess the public health impact for patients with bipolar disorder. During the follow-up, around more than 10000 suicide related events occurred. The incidence rate was significantly decreased by 14% during lithium treatment but not during valproate treatment. The difference in hazard ratios of suicide related events between lithium and valproate was statistically significant. Estimates of the population attributable fraction suggested that 12% of suicide related events could have been avoided if patients had taken lithium during the entire study.

Available treatments help a substantial proportion of patients, but are not beneficial for an estimated 40-50% of patients.

### SUMMARY OF THE INVENTION

Protein kinase C (PKC) appears to have a role in bipolar disorder. PKC is involved in controlling the function of proteins through the phosphorylation of hydroxyl groups of serine and threonine amino acid residues on these proteins, which are known to play a vital role in cell signalling pathways. It regulates multiple neuronal processes implicated in mood regulation. In current clinical practice, mood stabilizers and antidepressants have been shown to modulate the PKC pathway. Disrupted PKC activity has been found both in post-mortem brains and platelet from patients with mood disorders. Accumulating evidence suggests an imbalance of the PKC signalling system in mood disorders. Thus, PKC is considered as a novel molecular target for the development of innovative medicine for bipolar disorder.

Targeting the PKC signalling pathway for bipolar disorder can improve the patient compliance, when therapeutic dose monitoring is not required in patient, and such treatment can provide significant improvement in mania and depression.

Endoxifen is a non-steroidal selective estrogen receptor modulator (SERM) of the triphenylethylene group. It is an active metabolite of tamoxifen and has been found to be effective in patients that have failed previous hormonal therapies (tamoxifen, aromatase inhibitors, and fulvestrant). The prodrug tamoxifen is metabolized by the CYP2D6 enzyme to produce afimoxifene (4-hydroxytamoxifen) and endoxifen. The chemical name of endoxifen citrate is (Z)-1-(4-Hydroxyphenyl)-1-{4-[2-(monomethylamino)ethoxy]phenyl}-2-phenyl-1-butene citrate. The empirical formula of endoxifen citrate is C₂₅H₂₇NO₂—C₆H₈O₇, and has following chemical structure as given below (formula I):

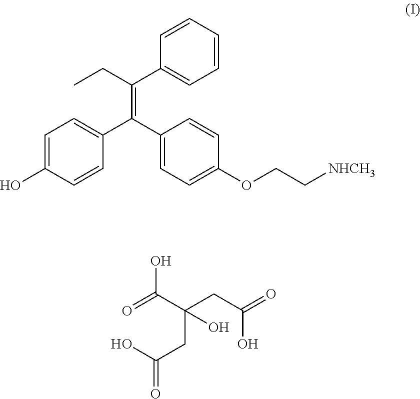 [SMILES: CC/C(=C(\c1ccc(O)cc1)c1ccc(OCCNC)cc1)c1ccccc1 ; O=C(O)CC(O)(CC(=O)O)C(=O)O]

The exact mechanism by which endoxifen exerts its therapeutic effects have not been established in bipolar disorder. However, the efficacy of endoxifen could be mediated through PKC. The PKC represents a family of enzymes highly enriched in brain, where it plays a major role in regulating both pre- and post-synaptic aspects of neurotransmission. Excessive activation of PKC results in symptoms related to bipolar disorder. The PKC signalling pathway is clearly a target for the actions of two structurally dissimilar antimanic agents—lithium and valproate. Tamoxifen, a widely used breast cancer drug is also known to inhibit PKC and demonstrate antimanic properties in human. Endoxifen exhibited four-fold higher potency compared to tamoxifen in inhibiting the PKC activity and is not dependent on the isozyme cytochrome P450 2D6 (CYP2D6) for action on the target tissues. Endoxifen is a PKC inhibitor and is effective in the treatment of bipolar disorder. Further, endoxifen has a broad therapeutic index as compare to divalproex sodium.

An object of the present disclosure is to provide a method for maintaining a therapeutically effective concentration of endoxifen for treatment of bipolar I disorder in a patient.

Another object of the present disclosure is to provide a method for maintaining a therapeutically effective concentration of endoxifen for treatment of bipolar I disorder in a patient, wherein the patient has manic episodes with or without mixed features.

Another object of the present disclosure is to provide a method for maintaining a therapeutically effective concentration of endoxifen for treatment of bipolar I disorder in a patient, wherein the patient has depression or associated with depressive episodes.

Another object of the present disclosure is to provide a method for maintaining a therapeutically effective concentration of endoxifen for the treatment of patient with bipolar I disorder, wherein the method comprises administrating a dose of 2 mg to 16 mg of endoxifen citrate in an enteric coated tablet once per day to patient in need thereof.

Another object of the present disclosure is to provide a method for maintaining a therapeutically effective concentration of endoxifen for the treatment of patient with bipolar I disorder, wherein the method comprises administrating a dose of 2 mg to 16 mg of endoxifen citrate in an enteric coated tablet once per day for at least 21 days to patient in need thereof.

Another object of the present disclosure is to provide a method for managing or decreasing a risk of suicidal behavior in patient undergoing treatment of bipolar I disorder, wherein the said method comprises maintaining the therapeutically effective concentration of endoxifen administrating a dose of 2 mg to 16 mg of endoxifen citrate in an enteric coated tablet once per day for at least 21 days to patient in need thereof.

Another object of the present disclosure is to provide a method for managing or decreasing a risk of suicidal behavior in patient undergoing treatment of bipolar I disorder, wherein the said method comprises maintaining the therapeutically effective concentration of endoxifen by administrating a dose of 2 mg to 16 mg of endoxifen citrate in an enteric coated tablet once per day for at least 21 days to patient in need thereof, wherein the risk of suicidal behavior in a patient is substantially less.

Another object of the present disclosure is to provide a method for managing or decreasing a risk of suicidal behavior in patient undergoing treatment of bipolar I disorder, wherein the said method comprises maintaining the therapeutically effective concentration of endoxifen by administrating a dose of 2 mg to 16 mg of endoxifen citrate in an enteric coated tablet once per day for at least 21 days to patient in need thereof, wherein the risk of suicidal behavior in a patient is zero.

Another object of the present disclosure is to provide a method for managing or decreasing a risk of suicidal behavior in patient undergoing treatment of bipolar I disorder, wherein the said method comprises maintaining the therapeutically effective concentration of endoxifen by administrating a dose of 2 mg to 16 mg of endoxifen citrate in an enteric coated tablet once per day for at least 21 days to patient in need thereof and that has progressed during or after treatment with at least one drug(s) selected from lithium, valproate, carbamazepine or an antipsychotic.

Another object of the present disclosure is to provide a method for managing or decreasing a risk of suicidal behavior in patient undergoing treatment of bipolar I disorder, wherein the said method comprises maintaining the therapeutically effective concentration of endoxifen by administrating a dose of 2 mg to 16 mg of endoxifen citrate in an enteric coated tablet once per day for at least 21 days to patient in need thereof and that has progressed during or after treatment with at least one drug(s) selected from lithium, valproate, carbamazepine or an antipsychotic, wherein the risk of suicidal behavior in a patient is substantially less.

Another object of the present disclosure is to provide a method for managing or decreasing a risk of suicidal behavior in patient undergoing treatment of bipolar I disorder, wherein the said method comprises maintaining the therapeutically effective concentration of endoxifen by administrating a dose of 2 mg to 16 mg of endoxifen citrate in an enteric coated tablet once per day for at least 21 days to patient in need thereof and that has progressed during or after treatment with at least one drug(s) selected from lithium, valproate, carbamazepine or an antipsychotic, wherein the risk of suicidal behavior in a patient is zero.

The objects disclosed herein are directed to a method for managing or decreasing a risk of suicidal behavior in patient undergoing treatment of bipolar I disorder, wherein the said method comprises maintaining the therapeutically effective concentration of endoxifen by administrating a dose of 2 mg to 16 mg of endoxifen citrate in an enteric coated tablet once per day for at least 21 days to patient in need thereof.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly throughout the specification and claims unless an otherwise expressly set out definition provides a broader definition.

For the purposes of the present disclosure, any ranges given include both the lower and the upper end points of the range. Ranges given should be considered approximate, unless specifically stated.

The term “C-SSRS” refers to Columbia-Suicide Severity Rating Scale.

The term “PP population” refers to per protocol population to those population who completed the study without major protocol violations/deviations.

The term “EOT” refers to end of treatment.

The term “therapeutically effective concentration” refers to a concentration of endoxifen in plasma which is sufficient to decrease or prevent or cure the symptoms associated with a medical condition or infirmity or to normalize body functions in disease or disorders that result in impairment of specific bodily functions.

The term “enteric coating” refers to any pharmaceutically acceptable coating preventing the release of the active agent in the stomach and sufficiently disintegrating in the intestine tract (by contact with approximately neutral or alkaline intestine juices) to allow the resorption of the active agent through the walls of the intestinal tract. The enteric coating remains intact in the acidic environment of the stomach and then solubilize in the more alkaline environment of the small intestine. Generally speaking, enteric coating helps in preventing gastric mucosal irritation and can be used for acid labile drugs which gets denatured in acidic medium.

In one embodiment the present application provides a method for maintaining a therapeutically effective concentration of endoxifen for treatment of bipolar I disorder in a patient.

In another embodiment, the present application provides a method for maintaining a therapeutically effective concentration of endoxifen for treatment of bipolar I disorder in a patient, wherein the patient has manic episodes with or without mixed features.

In another embodiment the present application provides a method for maintaining a therapeutically effective concentration of endoxifen for treatment of bipolar I disorder in a patient, wherein the patient has depression or associated with depressive episodes.

In another embodiment the present application provides a method for maintaining a therapeutically effective concentration of endoxifen for the treatment of patient with bipolar I disorder, wherein the method comprises administrating a dose of 2 mg to 16 mg of endoxifen citrate in an enteric coated tablet once per day for at least 21 days to patient in need thereof.

In another embodiment the present application provides a method for maintaining a therapeutically effective concentration of endoxifen for the treatment of patient with bipolar I disorder, wherein the method comprises administrating a dose of 2 mg to 16 mg of endoxifen citrate in an enteric coated tablet once per day for at least 21 days to patient in need thereof.

In another embodiment the present application provides a method for managing or decreasing a risk of suicidal behavior in patient undergoing treatment of bipolar I disorder, wherein the said method comprises maintaining the therapeutically effective concentration of endoxifen by administrating a dose of 2 mg to 16 mg of endoxifen citrate in an enteric coated tablet once per day for at least 21 days to patient in need thereof.

In another embodiment the present application provides a method for managing or decreasing a risk of suicidal behavior in patient undergoing treatment of bipolar I disorder, wherein the said method comprises maintaining the therapeutically effective concentration of endoxifen by administrating a dose of 2 mg to 16 mg of endoxifen citrate in an enteric coated tablet once per day for at least 21 days to patient in need thereof, wherein the risk of suicidal behavior in a patient is substantially less.

In another embodiment the present application provides a method for managing or decreasing a risk of suicidal behavior in patient undergoing treatment of bipolar I disorder, wherein the said method comprises maintaining the therapeutically effective concentration of endoxifen by administrating a dose of 2 mg to 16 mg of endoxifen citrate in an enteric coated tablet once per day for at least 21 days to patient in need thereof, wherein the risk of suicidal behavior in a patient is zero.

In another embodiment the present application provides a method for managing or decreasing a risk of suicidal behavior in patient undergoing treatment of bipolar I disorder, wherein the said method comprises maintaining the therapeutically effective concentration of endoxifen by administrating a dose of 2 mg to 16 mg of endoxifen citrate in an enteric coated tablet once per day for at least 21 days to patient in need thereof and that has progressed during or after treatment with at least one drug(s) selected from lithium, valproate, carbamazepine or an antipsychotic.

In another embodiment the present application provides a method for managing or decreasing a risk of suicidal behavior in patient undergoing treatment of bipolar I disorder, wherein the said method comprises maintaining the therapeutically effective concentration of endoxifen by administrating a dose of 2 mg to 16 mg of endoxifen citrate in an enteric coated tablet once per day for at least 21 days to patient in need thereof and that has progressed during or after treatment with at least one drug(s) selected from lithium, valproate, carbamazepine or an antipsychotic, wherein the risk of suicidal behavior in a patient is substantially less.

In another embodiment the present application provides a method for managing or decreasing a risk of suicidal behavior in patient undergoing treatment of bipolar I disorder, wherein the said method comprises maintaining the therapeutically effective concentration of endoxifen by administrating a dose of 2 mg to 16 mg of endoxifen citrate in an enteric coated tablet once per day for at least 21 days to patient in need thereof and that has progressed during or after treatment with at least one drug(s) selected from lithium, valproate, carbamazepine or an antipsychotic, wherein the risk of suicidal behavior in a patient is zero.

The embodiments disclosed herein are directed to a method for managing or decreasing a risk of suicidal behavior in patient undergoing treatment of bipolar I disorder, wherein the said method comprises maintaining the therapeutically effective concentration of endoxifen by administrating a dose of 2 mg to 16 mg of endoxifen citrate in an enteric coated tablet once per day for at least 21 days to patient in need thereof.

The embodiments described herein have been described by way of example only, and it is to be recognized that modifications thereto falling within the scope, and spirit of appended claims, and which would be obvious to a person skilled in the art based upon the disclosure herein, are also considered to be within the scope of this application.

Clinical Study Data:

Clinical study of an enteric coated tablet comprising endoxifen citrate formulation was carried out by administering to the patient doses of 2 mg to 16 mg of endoxifen citrate in an enteric coated tablet once per day for at least 21 days according to the present application.

Design of the Study:

This study was a multicenter, randomized, double-blind, double-dummy, active controlled, parallel study to assess the efficacy and safety of endoxifen enteric coated tablet 8 mg and divalproex sodium extended release tablet 1000 mg in patient of bipolar I disorders, this study also evaluated the risk of suicidal behavior in the patient and that has progressed during or after treatment with at least one drug(s) selected from lithium, valproate, carbamazepine or an atypical (except clozapine) or typical antipsychotic.

Total 110 patients were qualified for PP population.

The clinical trial study results are incorporated with different parameters as below,

TABLE 1
Proportion of patients with C-SSRS score at each visit (PP population)

| Day/Visit | Questions | Results | T (N = 110) | p-value |
|---|---|---|---|---|
| Day 0 | Q. 1 | YES | 0 (0.00%) | — |
| | | NO | 110 (100.00%) | |
| | Q. 2 | YES | 0 (0.00%) | — |
| | | NO | 110 (100.00%) | |
| Day 7 | Q. 1 | YES | 0 (0.00%) | — |
| | | NO | 110 (100.00%) | |
| | Q. 2 | YES | 0 (0.00%) | — |
| | | NO | 110 (100.00%) | |
| Day 14 | Q. 1 | YES | 0 (0.00%) | — |
| | | NO | 110 (100.00%) | |
| | Q.2 | YES | 0 (0.00%) | — |
| | | NO | 110 (100.00%) | |
| Day 21 | Q. 1 | YES | 0 (0.00%) | — |
| | | NO | 110 (100.00%) | |
| | Q. 2 | YES | 0 (0.00%) | — |
| | | NO | 110 (100.00%) | |

Treatment Specification: T-> Test Product.
Note 1:
Percentages are calculated based on the total number of patients in each treatment.
Note 2:
Q. 1 - Have you wished you were dead or wished you could go to sleep and not wake up ?
Q. 2 - Have you actually had any thoughts of killing yourself ?
p-value could not be calculated as there is no data for response YES in Q. 1 and Q. 2

At day 21 (EOT), endoxifen yielded the risk of suicidal behavior is zero in patient with bipolar I disorders.

Suicidal behavior was not reported during the clinical study.

1. A method for managing or decreasing a risk of suicidal behavior in a patient undergoing treatment of bipolar I disorder comprising:
maintaining a therapeutically effective concentration of endoxifen in the patient by administrating a dose of 2 mg to 16 mg of endoxifen citrate in an enteric coated tablet once per day for at least 21 days.

2. The method of claim 1, wherein the patient has manic episodes with mixed features.

3. The method of claim 1, wherein the patient has manic episodes without mixed features.

4. The method of claim 1, wherein the patient has depression.

5. The method of claim 1, wherein the patient associated with depressive episodes.

6. The method of claim 1, wherein the patient had the bipolar I disorder progress during or after treatment with at least one drug(s) selected from lithium, valproate, carbamazepine or an antipsychotic.
